(19) United States
(12) Patent Application Publication
(10) Pub. No.: US 2016/0175120 A1
(43) Pub. Date: Jun. 23, 2016

(54) FITTING SYSTEM FOR A PROSTHESIS

(71) Applicant: Michael Anthony KLAENE, Downingtown, PA (US)

(72) Inventor: Michael Anthony KLAENE, Downingtown, PA (US)

(21) Appl. No.: 14/979,049

(22) Filed: Dec. 22, 2015

Related U.S. Application Data
(60) Provisional application No. 62/095,127, filed on Dec. 22, 2014.

(51) Int. Cl. A61F 2/76

(52) U.S. Cl. CPC A61F 2/76; A61F 2002/7635; A61F 2002/769

(57) ABSTRACT

A fitting system for electronically representing both the static fitting and the dynamic movement of artificial prostheses is provided. The fitting system embodies a method of disposing a plurality of force sensing resistors along a prosthetic interface, wherein each force sensing resistor has an output element; noting a spatial relationship and a sequential relationship between each of the plurality of force sensing resistors; and providing a micro controller electrically interconnected between each output element and a display device, wherein the micro controller is configured to electrically represent a force output of each output element on the display device so that each force output is identified with the associated spatial relationship and the associated sequential relationship relative to the plurality of force sensing resistors.

### CROSS-REFERENCE TO RELATED APPLICATION

This application claims the benefit of priority of U.S. provisional application No. 62/095,127, filed 22 Dec, 2014, the contents of which are herein incorporated by reference.

### BACKGROUND OF THE INVENTION

The present invention relates to artificial prostheses for human limbs and, more particularly, to a fitting system for electronically representing both the static fitting and the dynamic movement of artificial prostheses.

The fitting and/or adjusting of a prosthetic element within a prosthetic socket is critical as this interface allows for comfortable weight-bearing, movement control and proprioception through a sturdy yet flexible connection that facilitates normal gait movement that does not bend under pressure. It is difficult, however, to determine the quality of the fit so that there is optimal surface bearing. Typically, prosthetists depend upon both observation and comments by the patient when fitting prosthetic elements, whereby guesswork is inherently involved in trying to achieve an optimal fit.

As can be seen, there is a need for a fitting system for electronically representing both the static fitting and the dynamic movement of artificial prostheses.

### SUMMARY OF THE INVENTION

In one aspect of the present invention, a method of fitting a prosthetic connector into a prosthetic socket includes disposing a plurality of force sensing resistors along a prosthetic interface, wherein each force sensing resistor has an output element; noting a spatial relationship and a sequential relationship between each of the plurality of force sensing resistors; and providing a micro controller electrically interconnected between each output element and a display device, wherein the micro controller is configured to electrically represent a force output of each output element on the display device so that each force output is identified with the associated spatial relationship and the associated sequential relationship relative to the plurality of force sensing resistors.

In another aspect of the present invention, the prosthetic interface includes an interface of the prosthetic connector and the prosthetic socket.

In yet another aspect of the present invention, each force output and identified associated spatial relationship and the associated sequential relationship includes a pressure value.

In yet another aspect of the present invention, the method further includes adjusting the fitting of the connector into the prosthetic socket in conjunction with relative pressure values.

In yet another aspect of the present invention, relative pressure values define a relative tightness of fit between the prosthetic connector and the prosthetic socket.

These and other features, aspects and advantages of the present invention will become better understood with reference to the following drawings, description and claims.

### BRIEF DESCRIPTION OF THE DRAWINGS

FIG. 1 is a schematic view of an exemplary embodiment of the present invention; and

FIG. 2 is a flow chart of an exemplary embodiment of the present invention.

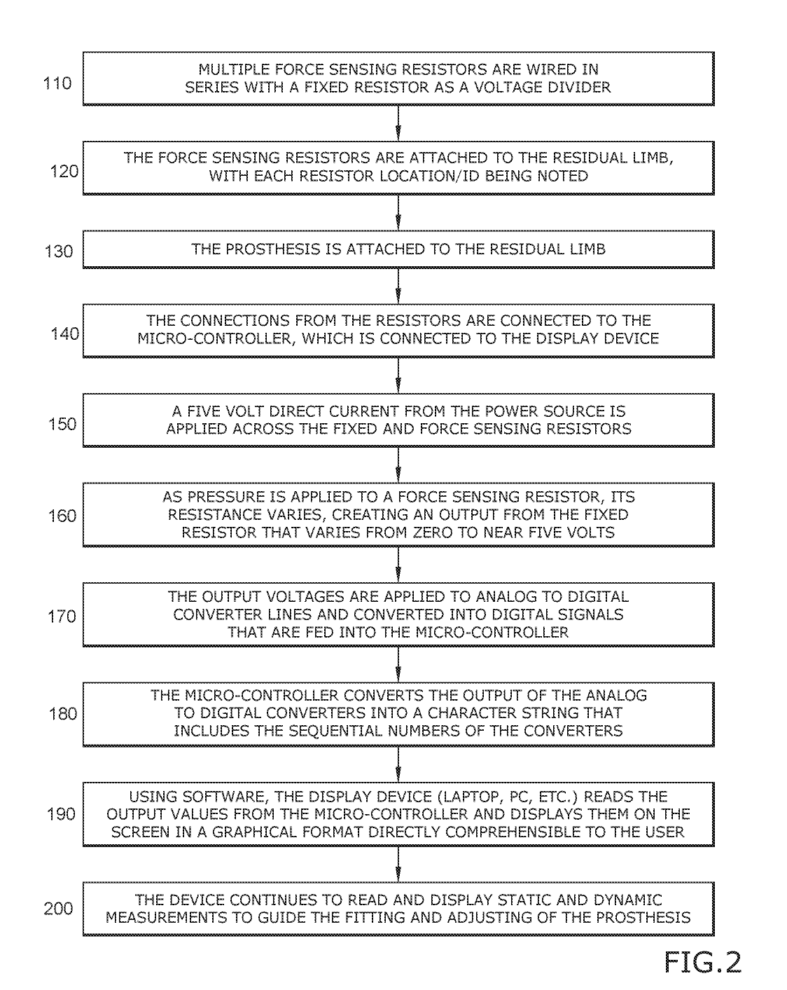

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a fitting system for electronically representing both the static fitting and the dynamic movement of artificial prostheses. The fitting system embodies a method of disposing a plurality of force sensing resistors along a prosthetic interface, wherein each force sensing resistor has an output element; noting a spatial relationship and a sequential relationship between each of the plurality of force sensing resistors; and providing a micro controller electrically interconnected between each output element and a display device, wherein the micro controller is configured to electrically represent a force output of each output element on the display device so that each force output is identified with the associated spatial relationship and the associated sequential relationship relative to the plurality of force sensing resistors.

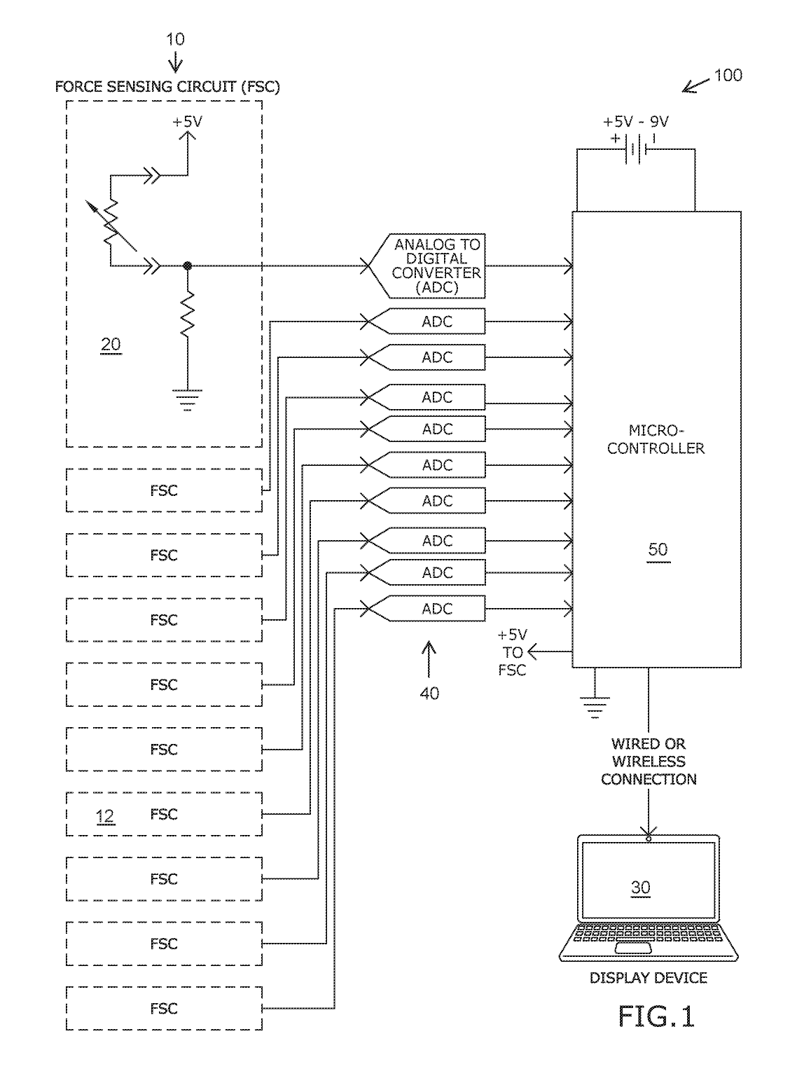

Referring to FIG. 1, the present invention may include a fitting system 100 for electronically representing both the static fitting and the dynamic movement of artificial prostheses. Prosthetic elements, such as prosthetic limbs, joints and other body parts, provide a prosthetic connector adapted to attach to prosthetic sockets, which are situated on the relevant stump of the prosthetic user. These prosthetic connector-sockets “interfaces” define the optimal fit critical to a successful prosthetic experience.

The fitting system 100 embodies a method of providing electronically representations for both the static fitting and the dynamic movement of artificial prostheses along the prosthetic interfaces. The fitting system 100 may include at least one circuitry assembly 10 disposed along the prosthetic connector and socket interface (or “prosthetic interface”). The circuitry assemblies 10 may be disposed along the prosthetic connector, along the prosthetic socket, or both. Each circuitry assembly 10 may be adapted to measure the pressure at a plurality of locations along the prosthetic interface.

Each circuitry assembly 10 may include a plurality of force sensing resistors (FSRs 12) electrically connected in series with a voltage divider circuit (VDC) and a power source 20. The force sensing resistors may alternatively be force and/or pressure sensors having output elements so long as they function in accordance with the FSR of the present invention as described herein. The VDC may be a fixed resistor. The power source 20 may be a five to nine volt DC power source or other sufficient potential provider. The power source 20 may be applied across the VDC with the positive side connected to the FSRs 12. The VDC have a plurality of output elements associated with each FSR 12. The fitting system 100 may provide a microprocessor 50 electrically interconnected between a display device 30 and at least one analog digital converter (ADC) 40 having a plurality of input elements, whereby the plurality of input elements are connected to the plurality of output elements. It should be noted that electrical connections may be hardwired, wireless, or both.

The plurality of FSRs 12 may be disposed along the prosthetic interface. Each FSR 12 may be adapted so that as pressure is applied to the FSR 12 its resistance varies. In certain embodiments, the resistance may vary from greater than 10M ohms to about 400 ohms. This results in an output voltage from each of the plurality of output elements of the VDC for an associated FSR 12 of the plurality of the FSRs 12, wherein each output voltage varies from zero volts (no pressure) to non-zero volts (as a function of pressure applied to the associated FSR and the power source 20). In other words, the change in resistance may be converted to a directly proportional output voltage.

The plurality of output voltages are then applied to the plurality of input elements of the ADC 40, wherein the ADC is adapted to convert such output voltages into a representative digital value than can be processed by the microprocessor 50.

The microprocessor 50 is adapted to read the output value of each of the ADC 40 in turn and converts it into an ASCII character string that also includes the sequential number for each ADC 40 input element. The resulting string of characters is the ADC pin number having an equal sign “=” followed by converted ADC value and the CR/LF sequence number.

The display device 30 is adapted to electronically represent and/or displays the plurality of ADC pin numbers on a screen in such a way as the results are directly understandable by the user.

Accordingly, the applied pressure for each FSR is simultaneously represented, wherein the sequential and thus spatial relationship between the applied pressures (“relative pressure”) is at least graphical represented on the display device 30, so that the relative pressure along the prosthetic interface when fitting and/or adjusting the prosthetic elements into the prosthetic socket can be ascertained, comprehended and associated with actions of the fitting/adjusting process. Thus facilitating both static fitting and dynamic measurements when applying varying pressures during the fitting/adjusting process. These representations can be used to represent snug versus loose fits. By knowing where the fit is loose, through the use of standard spacing and the spatial arrangement of the FSRs 12, the optimal fit can be achieved. Relative tightness of the interface may be similarly found through the analysis of the pressure differences between the plurality of FSRs 12. Furthermore, if it is needed to visually “see” the relative tightness in the fit, the user may consult the (graphical) representation of the display device 30.

A method of the using the present invention may include the following. The fitting system 100 disclosed above may be provided. In step 110, multiple FSR 12 may be connected in series with a fixed resistor as a VDC. The FSR 12 may be attached to the prosthetic connector, socket and/or interface with each FSR 12 location and ID being noted, in step 120. The prosthetic elements may be attached to the prosthetic socket, in step 130. The connections from the FSR 12 are connected to the micro controller 50, which in turn is connected to the display device 30, in step 140. A power source 20 providing a five volt direct current is applied across the fixed resistor and the FSR 12, in step 150. As pressure is applied to a FSR 12 its resistance varies, creating an output from the fixed resistor that varies from zero to near five volts, in step 160. The output voltages are applied to the ADC input elements and converted to digital signals that are fed into the micro controller 50, instep 170. The micro controller 50 converts the output of the ADC 40 into a character string that includes the sequential number of the ADC 40, in step 180. The display device 30 may read the output values from the micro controller 50 and display/represents them in a graphical format directly comprehensible to the user, in step 190. The fitting system 100 continues to read and display static and dynamic measurements to guide the fitting and adjusting of the prosthetic connector in the prosthetic socket, in step 200.

Similarly, the fitting system 100 may be used to determine needed adjustments to have a new or improved prosthetic socket made. Likewise, any situation where there is a desire to simultaneously display pressure from multiple points would benefit from the present invention.

It should be understood, of course, that the foregoing relates to exemplary embodiments of the invention and that modifications may be made without departing from the spirit and scope of the invention as set forth in the following claims.

What is claimed is:

1. A method of fitting a prosthetic connector into a prosthetic socket, comprising:
disposing a plurality of force sensing resistors along a prosthetic interface, wherein each force sensing resistor comprises an output element;
noting a spatial relationship and a sequential relationship between each of the plurality of force sensing resistors; and
providing a micro controller electrically interconnected between each output element and a display device, wherein the micro controller is configured to electrically represent a force output of each output element on the display device so that each force output is identified with the associated spatial relationship and the associated sequential relationship relative to the plurality of force sensing resistors.

2. The method of claim 1, wherein the prosthetic interface comprises an interface of the prosthetic connector and the prosthetic socket.

3. The method of claim 1, wherein each force output and identified associated spatial relationship and the associated sequential relationship comprises a pressure value.

4. The method of claim 3, further comprising adjusting the fitting of the connector into the prosthetic socket in conjunction with relative pressure values.

5. The method of claim 4, wherein relative pressure values define a relative tightness of fit between the prosthetic connector and the prosthetic socket.

6. The method of claim 1, further comprising electrically connecting the plurality of force sensing resistors in series with a voltage divider circuit and a power source, wherein the voltage divider circuit has a circuit output element associated with each output element of the plurality of force sensing resistors.

7. The method of claim 6, further comprising electrically interconnecting at least one analog digital converter having a plurality of input elements between the voltage divider circuit and the micro processor, wherein the plurality of input elements are connected to each circuit output element.
